(19) United States
(12) Patent Application Publication
(10) Pub. No.: US 2026/0274789 A1
(43) Pub. Date: Sep. 17, 2026

(54) PROCESS FOR THE PREPARATION OF SULFAMIDE DERIVATIVES

(71) Applicant: ADAMA AGAN LTD., Ashdod (IL)

(72) Inventors: Marwan E. SIMAAN, Nazareth (IL); Sveta FRONTON, Modiin (IL); Mahesh MASNA, Hyderabad, Telangana (IN); Bijukumar Gopinathan PILLAI, Hyderabad, Telangana (IN); Alexander SHTERENBERG, Rehovot (IL)

(73) Assignee: ADAMA AGAN LTD., Ashdod (IL)

(21) Appl. No.: 19/471,309

(22) PCT Filed: Mar. 27, 2024
(86) PCT No.: PCT/IL2024/050312

§ 371 Date: Sep. 30, 2025

(30) Foreign Application Priority Data
Mar. 30, 2023 (IN) 202311023989

(51) Int. Cl. C07C 303/40; C07D 239/54

(52) U.S. Cl. CPC C07C 303/40; C07D 239/54

(57) ABSTRACT

The present invention provides a process for the preparation of a compound of formula (I), wherein: R¹ and R² each independently is (C₁-C₁₂)alkyl, (C₂-C₆)alkenyl, (C₃-C₈)cycloalkyl, aryl, heteroaryl, or R1 and R² together with the nitrogen atom to which they are attached form a 5-8 membered ring optionally substituted with one or more groups each independently selected from (C₁-C₁₂)alkyl, (C₃-C₈)cycloalkyl, aryl, and heteroaryl, and optionally further interrupted with one or more atoms each independently selected from —O— and —S—, wherein said alkyl, alkenyl, and cycloalkyl each independently is optionally interrupted with one or more groups each independently selected from —O— and —S—.

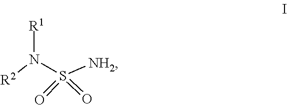

### TECHNICAL FIELD

The present invention provides a process for the preparation of sulfamide derivatives which are useful for the preparation of sulfamide-based compounds, such as sulfamide-based herbicidal active ingredients.

### BACKGROUND ART

Sulfamide derivatives, such as N,N-disubstituted sulfamides, are important chemical intermediates in organic synthesis.

Specifically, such derivatives can be utilized for the preparation of sulfamide-based compounds with multi substitutions which are commonly used in fields such as medicine and agricultural chemicals.

For instance, saflufenacil (2-chloro-5-[3,6-dihydro-3-methyl-2,6-dioxo-4-(trifluoromethyl)-1-(2H)pyrimidinyl]-4-fluoro-N-[[methyl(1-methylethyl)amino]sulfonyl]benzamide) which belongs to the class of substituted sulfamides, is a herbicide that is particularly useful for preplant applications and selective pre-emergence weed control in multiple crops. It is absorbed by foliage and roots with translocation in the apoplast and limited movement in the phloem. Saflufenacil is applied to foliage and is used for residual control of broad-leaved weeds, including glyphosate- and ALS-resistant biotypes.

Saflufenacil is applied pre-emergence in corn and sorghum, at 50-125 gram/hectare (g/ha); preplant for rapid foliar burn-down in soybeans, cereals, cotton, legumes; and post-directed in tree fruit and nuts, at 18-25 g/ha, and in sugarcane at 24.5-96 g/ha.

Saflufenacil belongs to the group of protoporphyrinogen oxidase (PPO) inhibitors, more specifically it is a pyrimidinedione-based PPO inhibitor and it has been described in WO 01/083459. Further processes for its preparation are described in WO 03/097589, WO 05/054208, WO 06/097589, WO 06/125746, WO 08/043835, and WO 08/043836.

WO 2001/083459 discloses the preparation of N,N-disubstituted sulfamides by, first, reacting chlorosulfonyl isocyanate with formic acid to obtain S-chlorosulfonamide. Then, the S-chlorosulfonamide is reacted with a secondary amine to obtain the desired product.

WO 03/097589 discloses a process for preparing sulfamoyl halides of primary or secondary amines, comprising: (i) reacting a primary or secondary amine with SO₃ or an SO₃ source in the presence of a tertiary amine such as α-picoline; and (ii) reacting the reaction mixture obtained in step (i) with a phosphorus halide such as phosphorus pentachloride. The obtained sulfamoyl halides can be further reacted with ammonia such as aqueous ammonia to obtain N,N-disubstituted sulfamides.

WO 2009/050120 discloses a process for the preparation of N,N-disubstituted sulfamides having the formula R¹R²N—S(O)₂—NH₂ by reacting sulfamoyl chloride having the formula R¹R²N—S(O)₂—Cl with ammonia, wherein said sulfamoyl chloride is used in the form of a solution in an inert solvent such as aromatic solvent. According to WO 2009/050120, the ammonia required for the reaction can be supplied in gaseous form or in the form of a solution, usually a non-aqueous solution, and provided that when the ammonia is supplied in the form of a solution, the solvent usually contains less than 1% protic components such as water.

There is an ongoing need for developing new and improved synthetic processes for preparing sulfamide derivatives, more specifically N,N-disubstituted sulfamides, thereby providing alternative routes that enable, e.g., to employ milder reaction conditions and overcome limitations in previously disclosed processes.

### SUMMARY OF INVENTION

The Experimental section herein shows the preparation of N-methyl-N-(1-methylethyl)aminosulfonamide. Specifically, sulfuryl chloride was added to a solution of N-isopropylmethyl amine in a solvent, more specifically toluene, to obtain N-isopropylmethylsulfamoyl chloride. Then, N-isopropylmethylsulfamoyl chloride was mixed with an ammonia-based solution, more specifically, a solution of ammonia in water or a solution of ammonia in methanol. The resulting solution was allowed to react and afford N-methyl-N-(1-methylethyl)aminosulfonamide.

In one aspect, the present invention thus relates to a process for the preparation of a compound of formula I

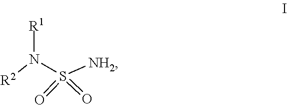

wherein:
R¹ and R² each independently is (C₁-C₁₂)alkyl, (C₂-C₆)alkenyl, (C₃-C₈)cycloalkyl, aryl, heteroaryl, or R¹ and R² together with the nitrogen atom to which they are attached form a 5-8 membered ring optionally substituted with one or more groups each independently selected from (C₁-C₁₂)alkyl, (C₃-C₈)cycloalkyl, aryl, and heteroaryl, and optionally further interrupted with one or more atoms each independently selected from —O— and —S—,
wherein said alkyl, alkenyl, and cycloalkyl each independently is optionally interrupted with one or more groups each independently selected from —O— and —S—,
said process comprising:
reacting a compound of formula II

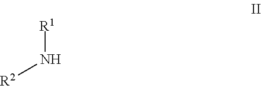

with sulfuryl chloride (SO₂CO₂) in a solvent, to thereby obtain a compound of formula III

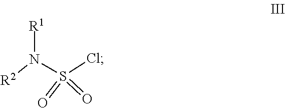

and
reacting the compound of formula III with an ammonia-based solution, to thereby obtain the compound of formula I.

In certain embodiments, disclosed herein is a process for the preparation of the compound of formula I as defined above, wherein said ammonia-based solution is a solution of ammonia in a protic solvent such as a solution of ammonia in water and a solution of ammonia in methanol.

In another aspect, the present invention provides a process for the preparation of a compound of formula IV

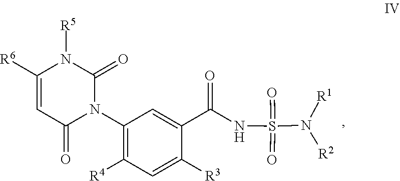

wherein:
R¹ and R² are each as defined above;
R³ and R⁴ each independently is H or halogen;
R⁵ is H or (C₁-C₆)alkyl; and
R⁶ is H, (C₁-C₆)alkyl, or (C₁-C₆)haloalkyl, said process comprising preparing the compound of formula I according to the process disclosed hereinabove and transforming said compound of formula I into the compound of formula IV.

### DETAILED DESCRIPTION

In one aspect, disclosed herein is a process for the preparation of N,N-disubstituted sulfamides of formula I, as defined above, said process comprising: (i) reacting a compound of formula II as defined above with sulfuryl chloride in a solvent, to thereby obtain a compound of formula III as defined above; and (ii) reacting the compound of formula III with an ammonia-based solution, to thereby obtain the compound of formula I.

The term “alkyl” typically means a linear or branched hydrocarbon group having. Particular alkyl groups are (C₁-C₁₂)alkyl such as methyl, ethyl, n-propyl, isopropyl, n-butyl, sec-butyl, isobutyl, tert-butyl, n-pentyl, 2,2-dimethylpropyl, n-hexyl, n-heptyl, n-octyl, and the like. Preferred are (C₁-C₈)alkyl, (C₁-C₆)alkyl, or (C₁-C₄)alkyl groups.

The term “alkenyl” typically means a linear or branched hydrocarbon group having one or more double bonds. Particular alkenyl groups are (C₂-C₆)alkenyl groups such as ethenyl, propenyl, 3-buten-1-yl, 2-ethenylbutyl, 3-octen-1-yl, and the like. Preferred are (C₂-C₄)alkenyl groups.

The term “cycloalkyl” means a mono- or bicyclic saturated hydrocarbyl group having, e.g., 3-8 carbon atoms such as cyclopropyl, cyclobutyl, cyclopentyl, cyclohexyl, cycloheptyl, and the like, that may be substituted, e.g., by one or more alkyl groups.

The term “aromatic ring” refers to an aromatic carbocyclic ring having, e.g., 6-14 carbon atoms, and consisting of a single ring or multiple rings either condensed or linked by a covalent bond. Non-limiting examples of aromatic rings include benzene, naphthalene, anthracene, naphthacene, phenanthrene, pyrene, chrysene, tetracene, and triphenylene. The term “aryl” as used herein denotes a univalent radical derived from an aromatic ring as defined herein by removal of hydrogen atom from any of the ring atoms. Preferred are (C₆-C₁₀)aryl groups.

The term “heteroaromatic ring” refers to a mono-, bi-, or poly-cyclic aromatic ring having, e.g., 4-12 atoms, and consisting of at least one carbon atom and at least one heteroatom selected from oxygen, sulfur or nitrogen. Preferred are 5- or 6-membered heteroaromatic rings. Non-limiting examples of heteroaromatic ring include thiophene, imidazole, pyridine, furan, pymole, oxazole, thiazole, purine, indole, pyrrole, pyrazine, isoquinoline, pyrazole, isoxazole, thiazole, isothiazole, pyrazine, pyrimidine, pyridazine, carbazole. The term “heteroaryl” as used herein refers to a univalent radical derived from a heteroaromatic ring as defined herein by removal of hydrogen atom from any of the ring atoms.

Each one of the alkyl, alkenyl, and cycloalkyl defined above may be interrupted with one or more atoms each independently selected from —O— and —S.

In certain embodiments, step (i) of the process of the invention comprises reacting the compound of formula II with sulfuryl chloride in said solvent, wherein the molar ratio between sulfuryl chloride and the compound of formula II is about 1:at least 1.8 (i.e., the number of moles of the compound of formula II is at least 1.8 times the number of moles of sulfuryl chloride), e.g., about 1:1.8, about 1:1.9, about 1:2.0, about 1:2.0 or more, respectively. According to the invention, the secondary amine of the formula II which is reacted with sulfuryl chloride in step (i) of the process disclosed herein may be added in a molar excess as compared to sulfuryl chloride, e.g., at least 1.8 molar equivalents relative to the moles of sulfuryl chloride, thereby acting as both the nucleophilic source (which reacts with sulfuryl chloride) and base (required to capture the HCl formed during said reaction), thus removing the need for an additional source of base that would add additional complexity the workup process. The excess of the secondary amine may be recovered and reused.

In certain embodiments, step (i) of the process of the invention comprises reacting the compound of formula II with sulfuryl chloride in said solvent in the presence of an organic base. Particular embodiments are those wherein said organic base is a tertiary amine-based base. Non-limiting examples of tertiary amine-based bases include a trialkyl amine such as trimethylamine, triethylamine, dimethylethylamine, and a combination thereof, an N-cycloalkyl-N,N-dialkylamine such as dimethylcyclohexylamine, diethylcyclohexylamine, and a combination thereof, benzyldimethylamine; pyridine; imidazole; and a combination thereof. According to the invention, step (i) of the process disclosed herein may comprise the use of an organic base such as a tertiary amine, e.g., to capture the HCl formed during said reaction, e.g., when the molar ratio between sulfuryl chloride and the compound of formula II is from about 1:0.8 to about 1:1.5, e.g., about 1:0.8, about 1:1, about 1:1.2-1.5, respectively.

In certain embodiments, the process of the present invention is a process as defined in any one of the embodiments above, wherein step (i) is carried out at a temperature ranging from about −15° C. to about 10° C., e.g., from about −10° C. to about 8° C., or from about −5° C. to about 0° C. or about 5° C.

In certain embodiments, the process of the present invention is a process as defined in any one of the embodiments above, wherein step (i) comprises the addition of sulfuryl chloride to a solution of the compound of formula II in said solvent, i.e., sulfuryl chloride is added, e.g., dropwise, to a solution of the compound of formula II in said solvent. In particular such embodiments, the addition of sulfuryl chloride is carried out over a period of at least 30 minutes, e.g., for at least 45 minutes, for at least 1 hour, for at least 1.5 hours, for at least 2 hours, for at least 2.5 hours, or for at least 3 hours; and/or at a temperature ranging from about −15° C. to about 10° C., e.g., from about −10° C. to about 8° C., or from about −5° C. to about 0° C. or about 5° C.

In certain embodiments, the process of the present invention is a process as defined in any one of the embodiments above, wherein the solvent used in step (i) is an aprotic organic solvent. Particular such embodiments are those wherein said aprotic organic solvent is a benzene-based solvent or a halogen-containing solvent. Non-limiting examples of benzene-based solvents include toluene, xylene, benzene, chlorobenzene, and mixtures thereof, and non-limiting examples of halogen-containing solvents include dichloromethane, dichloroethane, and mixtures thereof.

In certain embodiments, the process of the present invention is a process as defined in any one of the embodiments above, wherein the ammonia-based solution reacted with the compound of formula III as defined above in step (ii) is a solution of ammonia in a protic solvent. The term “protic solvent” as used herein refers to any solvent that contains a labile H⁺, and non-limiting examples of such solvents include water and alcohols such as methanol.

In particular such embodiments, the ammonia-based solution is aqueous ammonia, i.e., a solution of ammonia in water, e.g., having a concentration in the range of from about 15% to about 70%, e.g., from about 17% to about 65%, from about 19% to about 60%, from about 20% to about 55%, from about 20% to about 30%, from about 35% to about 55%, about 25%, or about 50%.

In other particular such embodiments, the ammonia-based solution is methanolic ammonia, i.e., a solution of ammonia in methanol, e.g., having a concentration in the range of from about 10% to about 30%, e.g., from about 12% to about 30%, from about 12% to about 25%, or about 7M, of ammonia in methanol.

As generally known, a one percent solution is defined as 1 gram of solute per 100 milliliters final volume, e.g., one gram of ammonia per 100 milliliters of protic solvent, as defined above, equals to a solution of ammonia in a protic solvent having a concentration of 1%.

In certain embodiments, the process of the present invention is a process as defined in any one of the embodiments above, wherein step (ii) is carried out at a temperature ranging from about −30° C. to about 10° C., e.g., from about −25° C. to about 8° C., from about −20° C. to about 7° C., from about −15° C. to about 6° C., or from about −10° C. to about 0° C. or 5° C.

In certain embodiments, the process of the present invention is a process as defined in any one of the embodiments above, wherein step (ii) further comprises the addition of a co-solvent. The term “co-solvent” as used herein refers to an organic solvent that is miscible in the solvent of the ammonia-based solution reacted in step (ii) of the process disclosed herein.

In particular such embodiments, the co-solvent is a polar organic solvent. Non-limiting examples of polar organic solvents include acetonitrile, acetone, dimethylformamide, dimethyl sulfoxide, and a combination thereof.

In certain embodiments, the process of the present invention is a process as defined in any one of the embodiments above, wherein step (ii) further comprises the addition of a co-solvent in an amount of up to about 30%, e.g., up to about 20%, or up to about 10%, for example from about 5% to about 25%, or from about 10% to about 20%, relative to the amount of the ammonia-based solution, by volume.

In certain embodiments, the process of the present invention is a process as defined in any one of the embodiments above, wherein the compound of formula III is obtained in step (i) in the form of a solution in said solvent; and said solution is reacted with said ammonia-based solution in step (ii). In particular such embodiments, the compound of formula II is reacted with sulfuryl chloride in said solvent to thereby obtain a solution of the compound of formula III in said solvent, wherein said solution is then washed with acidic aqueous solution, e.g., an aqueous HCl solution, to remove water miscible by-products such as unreacted compound of formula II in the form of a salt, e.g., an HCl salt of the compound of formula II, to thereby obtain an organic phase essentially consisting of the compound of formula III; and said organic phase is further reacted with the ammonia-based solution in step (ii). The term “essentially consisting of” as used herein with respect to the compound of formula III-containing organic phase obtained after acidic washing of the compound of formula III-containing solution obtained in step (i) means that said organic phase contains at least 80%, preferably at least 90% of the compound of formula III, by weight, i.e., said organic phase may contain up to about 20%, e.g., not more than 15%, preferably up to about 10%, of compounds other than the compound of formula III, by weight.

In certain embodiments, the process of the present invention is a process as defined in any one of the embodiments above, wherein the compound of formula III is obtained in step (i) in the form of a solution in said solvent; and said solution is concentrated under reduced pressure before being reacted with the ammonia-based solution in step (ii). In particular such embodiments, the compound of formula II is reacted with sulfuryl chloride in said solvent to thereby obtain a solution of the compound of formula III in said solvent, wherein said solution is then washed with acidic aqueous solution, e.g., an aqueous HCl solution, to remove water miscible by-products such as unreacted compound of formula II in the form of a salt, e.g., an HCl salt of the compound of formula II, to thereby obtain an organic phase essentially consisting of the compound of formula III; said organic phase is further concentrated under reduced pressure to thereby obtain the compound of formula III in a neat form; and said neat compound of formula III is reacted with the ammonia-based solution in step (ii).

In certain embodiments, the ammonia-based solution reacted in step (ii) of the process of the invention is a solution of ammonia in water having a concentration in the range of from about 15% to about 70%, e.g., from about 20% to about 55%.

Particular such embodiments are those wherein: (a) said ammonia-based solution is aqueous ammonia having a concentration in the range of from about 15% to about 35%, e.g., from about 20% to about 30%, or about 25%; (b) step (ii) further comprises the addition of a co-solvent; and/or (c) the compound of formula II is reacted with sulfuryl chloride in said solvent to thereby obtain a solution of the compound of formula III in said solvent, wherein said solution is then washed with acidic aqueous solution, e.g., an aqueous HCl solution, to remove water miscible by-products such as unreacted compound of formula II in the form of a salt, e.g., an HCl salt of the compound of formula II, to thereby obtain an organic phase essentially consisting of the compound of formula III; said organic phase is further concentrated under reduced pressure to thereby obtain the compound of formula III in a neat form; and said neat compound of formula III is reacted with the ammonia-based solution in step (ii).

Other particular such embodiments are those wherein: (a) said ammonia-based solution is a solution of ammonia in water having a concentration in the range of from about 40% to about 60%; and/or (b) the compound of formula II is reacted with sulfuryl chloride in said solvent to thereby obtain a solution of the compound of formula III in said solvent, wherein said solution is then washed with acidic aqueous solution, e.g., an aqueous HCl solution, to remove water miscible by-products such as unreacted compound of formula II in the form of a salt, e.g., an HCl salt of the compound of formula II, to thereby obtain an organic phase essentially consisting of the compound of formula III; said organic phase is further concentrated under reduced pressure to thereby obtain the compound of formula III in a neat form; and said neat compound of formula III is reacted with the ammonia-based solution in step (ii).

In certain embodiments, the ammonia-based solution reacted in step (ii) of the process of the invention is a solution of ammonia in methanol.

Particular such embodiments are those wherein: (a) step (ii) further comprises the addition of a co-solvent; and/or (b) the compound of formula III is obtained in step (i) in the form of a solution in said solvent; and said solution is reacted with said ammonia-based solution in step (ii).

In certain embodiments, the process disclosed herein, according to any one of the embodiments above, is for the preparation of a compound of the formula I, wherein R¹ and R² each independently is (C₁-C₆)alkyl such as methyl, ethyl, n-propyl, isopropyl, n-butyl, sec-butyl, isobutyl, tert-butyl, n-pentyl, 2,2-dimethylpropyl, and n-hexyl. In particular such embodiments, R¹ is methyl and R² is isopropyl.

The process disclosed herein, according to any one of the embodiments above, may be carried out with different efficiencies, depending on the reagents and conditions used. In other words, the product of this process may comprise different percentages of the desired compound I, as well as certain amounts of other compounds such as non-reacted starting material. Thus, in certain embodiments, the yield of the compound of formula I obtained by the process of the present invention is from about 40% to about 60%, from about 60% to about 80%, from about 80% to about 90%, or higher than 90%.

The N,N-disubstituted sulfamides of formula I prepared by the process disclosed herein according to any one of the embodiments above may be useful for the preparation of sulfamide-based compounds, e.g., sulfamide-based herbicidal active ingredients such as saflufenacil.

In another aspect, the present invention thus provides a process for the preparation of a compound of formula IV

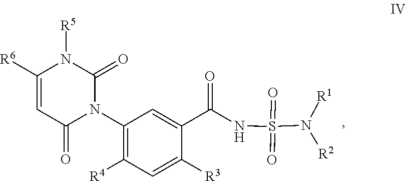

wherein:
R¹ and R² each independently each independently is (C₁-C₁₂)alkyl, (C₂-C₆)alkenyl, (C₃-C₈)cycloalkyl, aryl, heteroaryl, or R¹ and R² together with the nitrogen atom to which they are attached form a 5-8 membered ring optionally substituted with one or more groups each independently selected from (C₁-C₁₂)alkyl, (C₃-C₈)cycloalkyl, aryl, and heteroaryl, and optionally further interrupted with one or more atoms each independently selected from —O— and —S—,
wherein said alkyl, alkenyl, and cycloalkyl each independently is optionally interrupted with one or more groups each independently selected from —O— and —S—;
R³ and R⁴ each independently is H or halogen;
R⁵ is H or (C₁-C₆)alkyl; and
R⁶ is H, (C₁-C₆)alkyl, or (C₁-C₆)haloalkyl,
said process comprising preparing the compound of formula I according to the process disclosed herein, according to any one of the embodiments above, and transforming said compound of formula I into the compound of formula IV (also referred to herein as “a process for the preparation of the compound of formula IV”).

The term “halogen” as used herein refers to a halogen and includes fluoro, chloro, bromo, and iodo, but it is preferably fluoro or chloro.

The term “haloalkyl” typically means an alkyl as defined herein, substituted with one or more groups each independently selected from a halogen.

According to the invention, the process for the preparation of the compound of formula IV may include, in addition to the step of the preparation of the compound of formula I according to the process disclosed herein, any step(s) disclosed in the literature for the preparation of the compound of formula IV as defined hereinabove, e.g., as disclosed in WO 2001/083459.

In certain embodiments, the process for the preparation of the compound of formula IV disclosed herein is for preparing a compound of formula IV wherein R¹ and R² each independently is (C₁-C₆)alkyl such as methyl, ethyl, n-propyl, isopropyl, n-butyl, sec-butyl, isobutyl, tert-butyl, n-pentyl, 2,2-dimethylpropyl, and n-hexyl; R³ and R⁴ each independently is halogen such as Cl and F; R⁵ is H or (C₁-C₄)alkyl and R⁶ is (C₁-C₆)haloalkyl. Particular such embodiments are those wherein R¹ is methyl; R² is isopropyl; R³ is Cl; R⁴ is F; R⁵ is methyl; and R⁶ is CF₃ (saflufenacil).

Unless otherwise indicated, all numbers expressing, e.g., amounts of components or ratios between components, used in this specification, are to be understood as being modified in all instances by the term “about”. Accordingly, unless indicated to the contrary, the numerical parameters set forth in this specification are approximations that may vary by up to plus or minus 10% depending upon the desired properties to be obtained by the present invention.

The invention will now be illustrated by the following non-limiting Examples.

### EXAMPLES

### Materials and Methods

### Materials.

All reagents and starting materials were purchased from Sigma Aldrich, Acros Organics, and Alfa Aesar.

### Example 1. Synthesis of N-Methyl-N-(1-Methylethyl)Aminosulfonamide Using 50% Ammonia in Water

In a clean dry 0.5 L round bottom flask, toluene (120 mL, 5 vol) and N-isopropylmethyl amine (26.1 g, 2 eq) were charged under nitrogen atmosphere at 25-30° C., the mixture was stirred for 15 minutes and cooled to −5 to 0° C. Sulfuryl chloride (24 g, 1 eq) was then added dropwise into the reaction mass over 3.0 hours. Once the addition was complete, the reaction was maintained at that temperature for additional 30 minutes at which time full conversion to the intermediate N-isopropylmethylsulfamoyl chloride was observed. The reaction mixture was then maintained at 0° C. and washed twice with diluted HCl (1%, 37.5 mL, 1.5 vol) while maintaining temperatures between 0-5° C. The organic layer was then concentrated under reduced pressure (45-50° C., 40 mbar) to afford the crude intermediate as a light-yellow oil. In a separate 500 ml round bottom flask NH₄OH (50%) were charged (120 ml, 5V) and cooled to inner temperature of −10° C., to the cold solution the intermediate N-isopropylmethylsulfamoyl chloride was added dropwise over 5 minutes and the solution was mixed for 4 hours during which time it was warmed gradually to 0° C. and maintained at the that temperature. Once conversion was complete the solution was extracted with EtOAc (240 ml, 1OV) and the combined organic layers were reduced under pressure to afford the desired N-methyl-N-(1-methylethyl)aminosulfonamide product as a light yellow oil which crystalized upon sitting. The desired product was obtained with an isolated yield of 83%.

### Example 2. Synthesis of N-Methyl-N-(1-Methylethyl)Aminosulfonamide Using 25% Ammonia in Water

In a clean dry 0.5 L round bottom flask, toluene (120 mL, 5 vol) and N-isopropylmethyl amine (26.1 g, 2 eq) were charged under nitrogen atmosphere at 25-30° C., the mixture was stirred for 15 min and cooled to −5° C. Sulfuryl chloride (24 g, 1 eq) was then added dropwise into the reaction mass at 0-10° C. over 3.0 hours. Once the addition was complete, the reaction was maintained at that temperature for additional 30 minutes at which time full conversion to the intermediate N-isopropylmethylsulfamoyl chloride was observed. The reaction mixture was then cooled to 0° C. and washed twice with diluted HCl (1%, 37.5 mL, 1.5 vol) while maintaining temperatures at 5° C. The organic layer was then concentrated under reduced pressure (45-50° C., 40 mbar) to afford the crude intermediate as a light-yellow oil. In a separate 500 ml round bottom flask NH₄OH (25%) were charged (120 ml, 5V) and cooled to inner temperature of −10° C., to the cold solution the intermediate N-isopropylmethylsulfamoyl chloride was added dropwise over 5 minutes followed by acetonitrile (24 ml, 1V) and the solution was mixed for 4 hours during which it was warmed gradually to 0° C. and maintained at that temperature. Once conversion was complete the solution was extracted with EtOAc (240 ml, 10V) and the combined organic layers were reduced under pressure to afford the desired N-methyl-N-(1-methylethyl)aminosulfonamide product as a light yellow oil which crystalized upon sitting. The desired product was obtained with an isolated yield of 75%.

### Example 3. Synthesis of N-Methyl-N-(1-Methylethyl)Aminosulfonamide Using 7M Ammonia in Methanol

In a clean dry 0.5 L round bottom flask, toluene (120 mL, 5 vol) and N-isopropylmethyl amine (26.1 g, 2 eq) were charged under nitrogen atmosphere at 25-30° C., the mixture was stirred for 15 min and cooled to −5° C. Sulfuryl chloride (24 g, 1 eq) was then added dropwise into the reaction mass at 0-10° C. over 3.0 hours. Once the addition was complete, the reaction was maintained at that temperature for additional 30 minutes at which time full conversion to the intermediate N-isopropylmethylsulfamoyl chloride was observed. The reaction mixture was then cooled to 0° C. and washed twice with diluted HCl (1%, 37.5 mL, 1.5 vol) while maintaining temperatures at 5° C. The organic layer was then transferred to a 500 ml flask containing methanolic ammonia 7M (240 ml, 1OV) which was previously cooled to −5° C. Acetonitrile (24 ml, 1V) was added and the solution was mixed overnight during which it was warmed gradually to 0° C. and maintained at the that temperature. Once conversion was complete the reaction mixture was concentrated under reduced pressure and the resulting crude slurry was dissolved with 5V 1% HCl solution, extracted with EtOAc (240 ml, 1OV) and the combined organic layers were reduced under pressure to afford the desired N-methyl-N-(1-methylethyl)aminosulfonamide product as a light yellow oil which crystalized upon sitting. The desired product was obtained with an isolated yield of 63%.

1. A process for the preparation of a compound of formula I 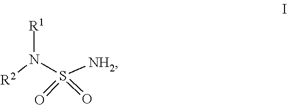
wherein:
R¹ and R² each independently is (C₁-C₁₂)alkyl, (C₂-C₆)alkenyl, (C₃-C₈)cycloalkyl, aryl, heteroaryl, or R¹ and R² together with the nitrogen atom to which they are attached form a 5-8 membered ring optionally substituted with one or more groups each independently selected from (C₁-C₁₂)alkyl, (C₃-C₈)cycloalkyl, aryl, and heteroaryl, and optionally further interrupted with one or more atoms each independently selected from —O— and —S—,
wherein said alkyl, alkenyl, and cycloalkyl each independently is optionally interrupted with one or more groups each independently selected from —O— and —S—,
said process comprising:
(i) reacting a compound of formula II 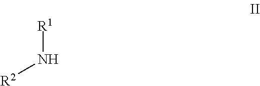
with sulfuryl chloride in a solvent, to thereby obtain a compound of formula III 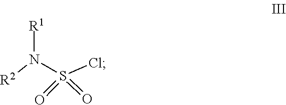
and
(ii) reacting the compound of formula III with an ammonia-based solution, to thereby obtain the compound of formula I.

2. The process of claim 1, wherein the molar ratio between sulfuryl chloride and the compound of formula II is 1:at least 1.8, respectively.

3. The process of claim 1, wherein step (i) comprises reacting the compound of formula II with sulfuryl chloride in said solvent in the presence of an organic base such as a tertiary amine-based base.

4. The process of claim 3, wherein said tertiary amine-based base is selected from the group consisting of a trialkyl amine such as trimethylamine, triethylamine, dimethylethylamine, and a combination thereof, an N-cycloalkyl-N,N-dialkylamine such as dimethylcyclohexylamine, diethylcyclohexylamine, and a combination thereof; benzyldimethylamine; pyridine; imidazole; and a combination thereof.

5. The process of any one of claims 1-4, wherein step (i) is carried out at a temperature ranging from about −15° C. to about 10° C., preferably from about −5° C. to about 5° C.

6. The process of any one of claims 1-5, wherein step (i) comprises the addition of sulfuryl chloride to a solution of the compound of formula II in said solvent.

7. The process of claim 6, wherein the addition of sulfuryl chloride is carried out over a period of at least 60 minutes; and/or at a temperature ranging from about −15° C. to about 10° C., preferably from about −5° C. to about 5° C.

8. The process of any one of claims 1-7, wherein said solvent is an aprotic organic solvent such as a benzene-based solvent and a halogen-containing solvent.

9. The process of claim 8, wherein said benzene-based solvent is selected from the group consisting of toluene, xylene, benzene, chlorobenzene, and a mixture thereof; and said halogen-containing solvent is selected from the group consisting of dichloromethane and dichloroethane.

10. The process of any one of claims 1-9, wherein said ammonia-based solution is a solution of ammonia in a protic solvent.

11. The process of claim 10, wherein said ammonia-based solution is a solution of ammonia in water having a concentration in the range of from about 15% to about 70%.

12. The process of claim 10, wherein said ammonia-based solution is a solution of ammonia in methanol.

13. The process of any one of claims 1-12, wherein step (ii) is carried out at a temperature ranging from about −30° C. to about 10° C., preferably from about −15° C. to about 0° C.

14. The process of any one of claims 1-13, wherein step (ii) further comprises the addition of a co-solvent.

15. The process of claim 14, wherein said co-solvent is a polar organic solvent such as acetonitrile, acetone, dimethylformamide, dimethyl sulfoxide, and a combination thereof.

16. The process of claim 14 or 15, wherein the co-solvent constitutes up to about 20% relative to the amount of the ammonia-based solution, by volume.

17. The process of any one of claims 1-16, wherein the compound of formula III is obtained in step (i) in the form of a solution in said solvent; and said solution is reacted with said ammonia-based solution in step (ii).

18. The process of any one of claims 1-16, wherein the compound of formula III is obtained in step (i) in the form of a solution in said solvent; and said solution is concentrated under reduced pressure before being reacted with said ammonia-based solution in step (ii).

19. The process of claim 1, wherein said ammonia-based solution is a solution of ammonia in water having a concentration in the range of from about 15% to about 70%.

20. The process of claim 19, wherein:
a) said ammonia-based solution is a solution of ammonia in water having a concentration in the range of from about 15% to about 35%;
b) step (ii) further comprises the addition of a co-solvent; and/or
c) the compound of formula III is obtained in step (i) in the form of a solution in said solvent; said solution is then washed with an acidic aqueous solution to thereby obtain an organic phase essentially consisting of the compound of formula III; said organic phase is further concentrated under reduced pressure to thereby obtain the compound of formula III in a neat form; and said neat compound of formula III is reacted with said ammonia-based solution in step (ii).

21. The process of claim 19, wherein:
a) said ammonia-based solution is a solution of ammonia in water having a concentration in the range of from about 40% to about 60%; and/or
b) the compound of formula III is obtained in step (i) in the form of a solution in said solvent; said solution is then washed with an acidic aqueous solution to thereby obtain an organic phase essentially consisting of the compound of formula III; said organic phase is further concentrated under reduced pressure to thereby obtain the compound of formula III in a neat form; and said neat compound of formula III is reacted with said ammonia-based solution in step (ii).

22. The process of claim 1, wherein said ammonia-based solution is a solution of ammonia in methanol.

23. The process of claim 22, wherein:
a) step (ii) further comprises the addition of a co-solvent; and/or
b) the compound of formula III is obtained in step (i) in the form of a solution in said solvent; and said solution is reacted with said ammonia-based solution in step (ii).

24. The process according to any one of claims 1-23, wherein R¹ and R² each independently is (C₁-C₆)alkyl such as methyl, ethyl, n-propyl, isopropyl, n-butyl, sec-butyl, isobutyl, tert-butyl, n-pentyl, 2,2-dimethylpropyl, and n-hexyl.

25. A process for the preparation of a compound of formula IV 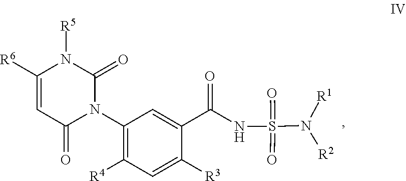
wherein:
R¹ and R² are each as defined in claim 1;
R³ and R⁴ each independently is H or halogen;
R⁵ is H or (C₁-C₆)alkyl; and
R⁶ is H, (C₁-C₆)alkyl, or (C₁-C₆)haloalkyl,
said process comprising preparing the compound of formula I according to any one of claims 1-24 and transforming said compound of formula I into the compound of formula IV.
